(19) United States
(12) Patent Application Publication
(10) Pub. No.: US 2017/0045450 A1
(43) Pub. Date: Feb. 16, 2017

(54) Method and Apparatus for Nondestructive Quantification of Cannabinoids

(71) Applicants: Chad Allen Lieber, Maplewood, MO (US); Randall James Kruep, Los Altos Hills, CA (US); Alexander James Makowski, Shamokin, PA (US)

(72) Inventors: Chad Allen Lieber, Maplewood, MO (US); Randall James Kruep, Los Altos Hills, CA (US); Alexander James Makowski, Shamokin, PA (US)

(21) Appl. No.: 14/824,017

(22) Filed: Aug. 11, 2015

(51) Int. Cl. G01N 21/64

(52) U.S. Cl. CPC G01N 21/645; G01N 33/0098

(57) ABSTRACT

An optically-based method and apparatus for monitoring a cannabis sample is provided. The method includes selecting a light source; selecting an optional optical filter; and applying the light source to illuminate a sample, wherein at least one of: light reflected from the sample, light transmitted through the sample, and light produced by fluorescence of the sample, is directed from the sample to the optical filter.

This application includes background from U.S. application Ser. No. 14/242,813 and claims the benefit of U.S. Provisional Application No. 62/031,971 filed Aug. 11, 2014, both of which are hereby incorporated by reference in their entirety for all purposes.

### BACKGROUND

Processing of cannabis often involves testing for its potency and other attributes for regulatory requirements and customer information processing. The attributes of the sample may be critical to the quality of the final product. Conventional methods for analyzing the products, such as removing a sample from a grow or production process, sending the sample to a testing facility remote from the production operations, and waiting for the sample analysis results, tend to be time consuming, invasive, and cumbersome. These techniques also tend to delay or disrupt operations. In addition, the conventional methods may be susceptible to sampling and processing errors and as such may not be reliably reproducible. Further, destruction of the sample after testing is often necessary, causing waste and disposal issues.

This application describes a system having an optical device and an appropriate method for using this optical device to nondestructively quantify cannabinoids using optical fluorescence in myriad sample types that overcomes one or more of these drawbacks of the prior art. It is within this context that the embodiments arise.

### SUMMARY

A method and related apparatus for monitoring a sample of cannabis product are provided. In some embodiments the method for monitoring a sample includes directing light from a light source to a sample, so as to illuminate the sample, and directing light from the illuminated sample to an optical measuring device. The method includes measuring at least a portion of a spectrum of the light from the illuminated sample via application of the optical measuring device and deriving information pertaining to a chemical property of the sample from the measured light.

In some embodiments, an optically-based method for monitoring a cannabis sample is provided. The method includes selecting a light source; selecting an optional optical filter; and applying the light source to illuminate a sample, wherein at least one of: light reflected from the sample, light transmitted through the sample, and light produced by fluorescence of the sample, is directed from the sample to the optical filter. In the case of ground sample or liquid extract of a sample, the method includes applying the optical filter to remove at least a portion of the light directed from the sample to the optical filter and measuring amplitude or intensity of light from the sample as filtered by the optical filter, for at least one wavelength of light. The method includes correlating the measured amplitude or intensity of light from the sample for the at least one wavelength of light with a chemical property of the sample. The method could also be applied in-line, by continuous measurement, by applying the steps detailed at a repetitive interval.

In some embodiments, an optically-based cannabis monitoring apparatus is provided. The apparatus includes a light source, an optional optical filter, and an optical measuring device selected from a group consisting of: optical detectors, including photodiodes, photodetectors, photodiode arrays, and charge coupled devices, and optical selection elements, selected from a group consisting of: gratings, prisms, diffractive elements, dielectric filters, and absorptive filters. The apparatus includes a machine, configured to perform actions including: illuminating a sample with the light source; filtering light from the sample with the optical filter so as to decrease, in light from the optical filter, intensity of light in at least one wavelength range related to light from the light source; and analyze, via application of the optical measuring device, the light from the optical filter as to wavelength and one of amplitude or intensity, wherein such analysis is applicable to a chemical property of the sample.

Other aspects and advantages of the embodiments will become apparent from the following detailed description taken in conjunction with the accompanying drawings which illustrate, by way of example, the principles of the described embodiments.

### BRIEF DESCRIPTION OF THE DRAWINGS

The described embodiments and the advantages thereof may best be understood by reference to the following description taken in conjunction with the accompanying drawings. These drawings in no way limit any changes in form and detail that may be made to the described embodiments by one skilled in the art without departing from the spirit and scope of the described embodiments.

FIG. 1 is a block diagram of an optically-based monitoring apparatus, applied to monitoring a sample in accordance with some embodiments.

FIG. 1A is a block diagram of a spectral engine suitable for use in an embodiment of the monitoring apparatus of FIG. 1 in accordance with some embodiments.

FIG. 2 is a block diagram of a further embodiment of the monitoring apparatus of FIG. 1 in accordance with some embodiments.

FIG. 3 illustrates a system that may be utilized for a characterized process, employing an embodiment of the monitoring apparatus of FIG. 1 in accordance with some embodiments.

FIG. 4 is a photometer device and apparatus that utilizes fluorescence of the cannabinoids for their quantification.

FIG. 4A is a representative hand-held photometer device in accordance with some embodiments of the present invention.

FIG. 5 is a representative fluorescence spectrum of a sample using the photometer device of FIG. 4.

FIG. 6 is a regression model for the sample measurement using the photometer device of FIG. 4.

FIG. 7 is illustrative of wavelength band-pass filters that could be used on the two preferred detectors of the photometer device of FIG. 4.

### DETAILED DESCRIPTION

The state of the art in cannabis chemical characterization entails established laboratory analytical techniques, including chromatographic (gas GC and liquid LC) and mass spectroscopic methods. While these methods provide accurate molecular characterization, they typically require sample preparation and/or destruction, the use of consumable preparatory materials, a skilled operator, and/or a waiting period of one or more minutes, and usually several or more minutes to obtain a result. An industry participant subject to regulatory oversight in legal markets must also endure additional cost and wait times for transport of samples to third party laboratories that must conduct the testing, in addition to wait times and business disruption while awaiting the testing results.

Optical techniques such as reflectance, absorption, or fluorescence possess several hallmark advantages over conventional analytical techniques for chemical characterization and quantification. Optical techniques are nonintrusive to the sample, which allows measurement without altering the chemical content or causing any physical change to the sample. Fluorescence measurements uniquely have very high specificity, relying on the absorption of specific wavelengths of light by the sample, and the resulting fluorescence at a longer wavelength. Fluorescence measurements are known to be very sensitive and selective. Optical techniques are also very rapid, allowing sample measurements typically less than one second. Optical techniques also possess molecular specificity, such that individual molecular compounds of interest can be identified uniquely, typically without any further sample preparation. Optical techniques can also provide automated characterization or quantification, allowing their use by unskilled personnel or with minimum training. Finally, devices based on optical techniques require little or no maintenance over their operating lifetime.

Devices based on the described optical techniques can be separated into two primary categories: spectral devices that utilize tens to thousands of individual wavelengths across a spectral range, and photometer devices that utilize one to ten discrete wavelength bands. These devices can be generally described, respectively, as spectrometer and photometer devices.

Spectrometer devices rely on the detection of a continuous range of optical intensities across a portion of the optical wavelength spectrum. These devices typically employ a broadband illumination source such as an incandescent lamp or multiple light-emitting diodes (LEDs) to illuminate the sample with all of the wavelength range of interest in a single exposure. Alternately, a narrow band source such as a laser can be used as an illumination source, which can then be wavelength scanned to permit illumination across the wavelength range of interest. Typically, the illumination source is directed to the sample using some delivery optics, including lenses, prisms, filters, optical fibers, or optical waveguides. The light impinges on the sample where a portion of it is absorbed, a portion is scattered or reflected, and a minor fraction undergoes a quantum mechanical alteration leading to emission of fluorescence or Raman scattering. The light coming from the sample is then typically collected by optical elements such as lenses, prisms, filters, optical fibers, or optical waveguides, then directed to a spectrometer. The spectrometer contains two primary elements: a wavelength separating element such as a grating or prism, and a light detector. In a spectrometer, this detector is typically a multi-element device such as a CCD or photodiode array to permit simultaneous detection of all wavelengths in a single acquisition and without any moving optical elements. Alternately, Fourier-transform based devices may be utilized which rely on optical interference generated by a moving reflector and a single optical detector. Other embodiments use some combination of scanning elements or dispersing elements to acquire the multiple spectral points in simultaneous or successive manner.

Photometer devices rely on the detection of a limited number of optical intensities at predetermined wavelengths. As such, these devices are typically less complex and less costly than spectrometer devices. Though both types of devices may share similar delivery and collection optics to interface the light with the sample, the illumination source and detectors are typically much simpler in the photometer. The illumination source(s) in a photometer are typically a discrete emitter such as a LED, laser diode, or laser, to enable illumination only within a narrow wavelength range. Alternately, an incandescent source can be employed with wavelength blocking filters to limit its emission to discrete wavelength bands. The detector(s) in a photometer are typically single-element photodiodes or similar, which may have wavelength suppressing filters installed to enable optical detection at discrete wavelength bands.

Whether a spectrometer or photometer device, the generation of a meaningful constituent output relies on prior modeling and chemometrics to relate the optical signal to chemical content or quantity. For chemical speciation, this process entails the acquisition of optical information from the purified chemical species of interest, such that its unique optical characteristics can be used as a “fingerprint” that can be used to detect its presence in later analyses of chemical mixtures that may contain this species of interest. For quantification, training sets with various quantities of the species of interest are first measured by the optical device, while the training set samples are then accurately quantified via an accepted “gold-standard” reference measurement with traceable methodology, such as gas or liquid chromatography or mass spectroscopy. Statistical treatment of the optical information is typically necessary to develop algorithms or models which relate the optical information to chemical species quantity. This treatment is often multivariate in nature, and may involve techniques such as simple or multiple regressions, principal components analysis, partial least squares analysis, or others. Once these models are developed, they can be integrated into the optical device such that its measurements can be fed into this model so a constituent or quantity of interest can be returned, without further input from the user.

The prior art systems and methods of accurate measurement of species within cannabinoids have many drawbacks including being lengthy, costly, inconvenient, remote, non-portable, and require skilled operators, and preparation of and destruction of the cannabinoids and the samples. A major limitation of traditional analytical tools for evaluating cannabis potency is that the plant samples must be subjected to an extraction to remove the cannabinoids from the complex chemical matrix that composes the whole plant. These extractions often employ solvents, such as chloroform and methanol, which are health hazards, and require distinct safety precautions to be implemented. Additionally, if GC is used to measure potencies, the molecules must be volatile, i.e. readily converted from a liquid to gaseous state. This means that the acidic forms of THC and CBD cannot be measured with GC, unless they are derivatized with a molecule that makes them more volatile. This added sample preparation requirement increases the skill requirement of the operator, decreases the number of samples a researcher can measure in time, and thus increases the cost of testing. As previously mentioned, GC destroys the sample, and retrieval of the compounds of interest in LC would require costly and laborious purification.

Each participant of the legal cannabis industry (growers, dispensaries, regulators, marijuana-infused product manufacturers) has an important need to ensure accuracy and consistency of their cannabis potency measurements, this necessity has been hindered by the inherent obstacles of, and omissions to current testing methods. Specifically, until now, nearly all testing must be conducted off-site of a business or regulators' operations. This burden causes delay, and because of this delay, one's operations are prone to error or inconsistency. Safety is an issue, as the need to transport cannabis samples having such a high monetary and black market value can present a risk to the transport agent. The sampling of cannabis plants in the field can lead to contamination of the samples to be evaluated, which can result in errors in the analysis. This contamination may stem from, for example, extraneous debris adhering to the harvested plant material. Most notably, if on-site testing was available on a cost-effective basis, this would enable quality control and production efficiencies to increase considerably. Additionally, costs, delay and transport risks could be significantly reduced.

Advantageously, the present invention enables operators to measure samples on-site, using a portable, semi-portable apparatus, and hand-held device without requiring transport to a remote laboratory, wait times for the results, and destruction of the sample after testing. The facility of the sample preparation using the optical based method and apparatus of the present invention lowers the skill requirement and time required for testing, and thus enables skilled personnel more time to spend evaluating samples, rather than time-consuming, often toxic preparatory steps. The apparatus can measure samples in any form (i.e., solids, oils, waxes) for a diverse assortment of cannabis-based products including flowers, kief, hash, oil, waxes, concentrates, etc. Whole buds can be measured, however it is a preferred embodiment that the samples be homogenized through grinding, to provide a more thorough and accurate measurement of cannabinoid levels throughout the bud. For example, trichomes contain high THC levels, and if the user honed in on this area of the plant, falsely high THC contents may be reported. Likewise, if the sample contains stem material, and the NIR light probes this region of the sample, anticipated cannabinoid levels will be skewed.

The present invention is a system and apparatus having an optical device or apparatus and an appropriate method for using this optical device to nondestructively quantify cannabinoids in myriad sample types that overcomes one or more of these drawbacks of the prior art.

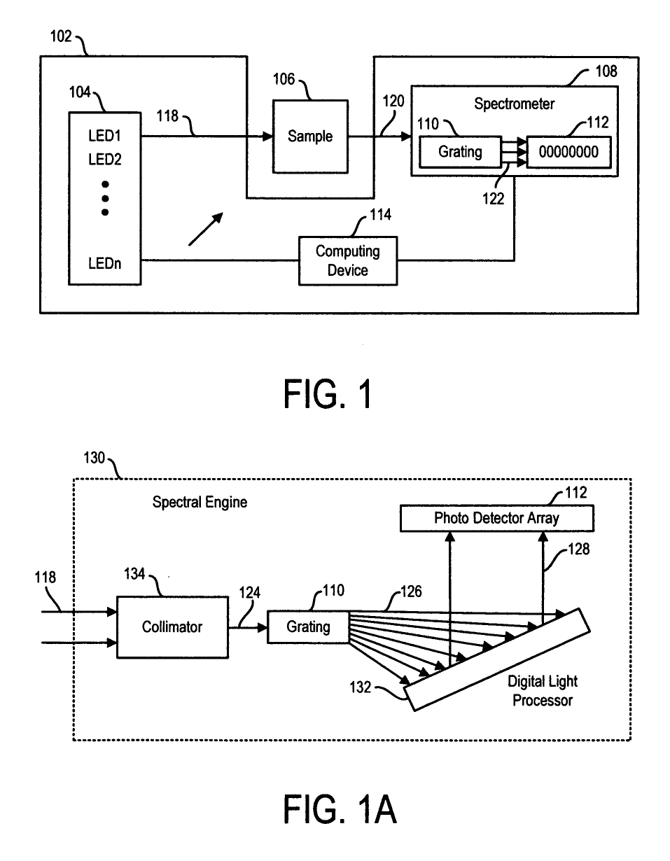

FIG. 1 is a block diagram of an optically-based monitoring apparatus 102, applied to monitoring a sample 106. The monitoring apparatus 102 provides a plurality of LEDs (LED₁-LEDₙ) that operate as a light source 104. In variations, other types of light sources can be used, such as monochromatic light sources, narrowband light sources, wideband light sources, a light source with an optical filter (which could be narrowband or wideband), a tungsten light source, a halogen light source, ultraviolet light sources, infrared or near infrared light sources, a xenon lamp, a deuterium lamp, glowing metal for thermal emission of infrared, a white light source, etc.

It should be appreciated that the use of an LED as a light source 104 provides a discrete wavelength of light 118 (as LEDs are monochromatic) and eliminates the use of a selection filter. LEDs are available in a wide range of visible colors and also ultraviolet. Visible color LEDs can be used for embodiments of the monitoring apparatus 102 making use of light reflection or transmission by a sample, and ultraviolet LEDs can be used for embodiments of the monitoring apparatus 102 making use of light induced fluorescence of the sample. The plurality of LEDs may include multiple copies of the same LED (i.e., all of the LEDs emit the same wavelength) or different LEDs (each emitting different wavelengths) or some combination of both. In some embodiments, the plurality of LEDs is embodied on a puck (or circuit board) as an array of LEDs. Or, a single LED could be used. In embodiments using one or more preferably ultraviolet LEDs, the LED excites the sample and causes an emission of light if the material is fluorescent. In a material that fluoresces, the emission wavelength will be longer, i.e., at lower energy, than the excitation wavelength.

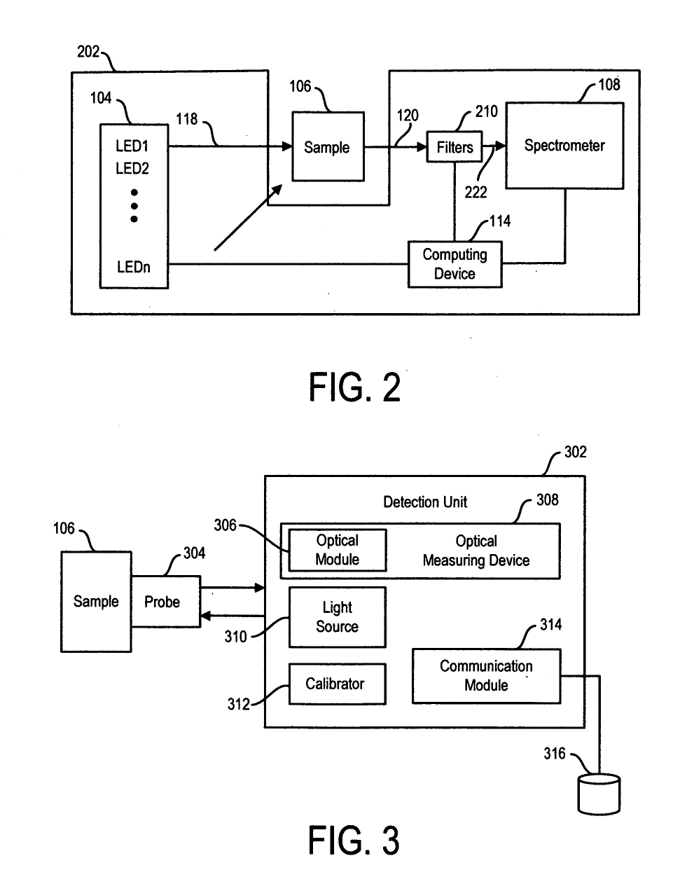

The sample 106 of FIGS. 1 and 2 may be any cannabis product in a form of suitable solid or liquid material or slurry or suspension. It should be appreciated that the excitation and the emission may occur through the same sampling window. For example, a probe providing the excitation light to the sample may also be configured to receive the emission light from the sample and deliver the light to a spectrometer 108 in some embodiments. In other embodiments, the probe provides light to the sample 104, and receives reflected, transmitted or emitted light from the sample.

With ongoing reference to FIG. 1, one embodiment of the optically-based monitoring apparatus 102 has a light source 104, a spectrometer 108, and the computing device 114. The spectrometer 108 includes a grating 110 and a photo diode array (PDA) 112. The grating 110 is an optical grating, which could be a transmission grating or a reflection grating, having a plurality of microscopic lines to direct different wavelengths of the excitation light toward respective regions of the PDA 112. The PDA 112 is a one dimensional pixel array in some embodiments. The pixel array may be composed of charged couple devices (CCD) or complementary metal oxide semiconductor (CMOS) devices in some embodiments. In further embodiments, the PDA 112 includes photodiodes or photo transistors. A computing device 114 is in communication with the spectrometer 108 and the LED array or other light source 104. The computing device 114 may control the activation of the different LEDs of the LED array and process the output of the raw data from the spectrometer 108. It should be appreciated that other configurations of spectrometers 108 may also be used. The alternative configurations for the spectrometers 108 may be designed to contemporaneously measure all of the emitted wavelengths of light from the sample and measure the full spectrum of the emission for each different LED excitation to build the EEM. It should be further appreciated that the computing device 114, in combination with various electronics components or electromechanical actuators, motors or other mechanisms for selecting and operating components in the monitoring apparatus 102, constitutes a configurable machine, and that other types of machines could be devised to perform these functions.

FIG. 1A is a block diagram of a spectral engine 130 suitable for use in an embodiment of the monitoring apparatus 102 of FIG. 1. The spectral engine 130 substitutes for or is a type of spectrometer 108 or optical measuring device. It should be appreciated that other types of optical measuring devices, such as a photodetector, a photodetector array, optical gratings, optical filters and so on can also be used. In the spectral engine, a collimator 134 collimates incoming light 118 from a sample 106 into a narrow beam 124. The narrow beam 124 is incident on a diffraction grating 110, which is herein shown as a transmission grating but could instead be a reflection grating. The diffraction grating 110 disperses light into a spectrum 126 of various intensities at various wavelengths. The spectrum 126 of light, originating from the sample 106, is selectively reflected by the digital light processor 132, which could be under control of the computing device 114. The digital light processor 132 could be a type of digital micro-mirror device, applying micro-electromechanical system (MEMS) technology. For example, the computing device 114 could set specific mirrors on the digital light processor 132 to reflect light of wavelengths of interest, such as when a particular spectrum is expected or desired, or a particular peak or trough in the spectrum is to be selected for analysis. Or, the digital light processor 132 could be configured to activate mirrors in sequence so that the photodetector array 112 (or single photodetector) looks at only one range of wavelengths at a time. The grating 110 and collimator 134 may be internal to the digital light processor 132 in some configurations. Various lenses and mirrors can be applied to shape or direct the light as desired. The digital light processor 132 could be operated in conjunction with a selection of one of the light sources 104, thus acting as a replacement or substitute for a selective optical filter. For example, the digital light processor 132 could be operated to not reflect, i.e., to deselect, a range of wavelengths emitted by a narrow band one of the light sources 104. Or, the digital light processor 132 could be operated to reflect, i.e., to select, one or more ranges of wavelengths expected as peaks of fluorescence by a sample, or troughs of interest in selective absorption by a sample. The digital light processor 132 could also be operated in conjunction with one or more selective optical filters, for example to obtain increases in accuracy and readings.

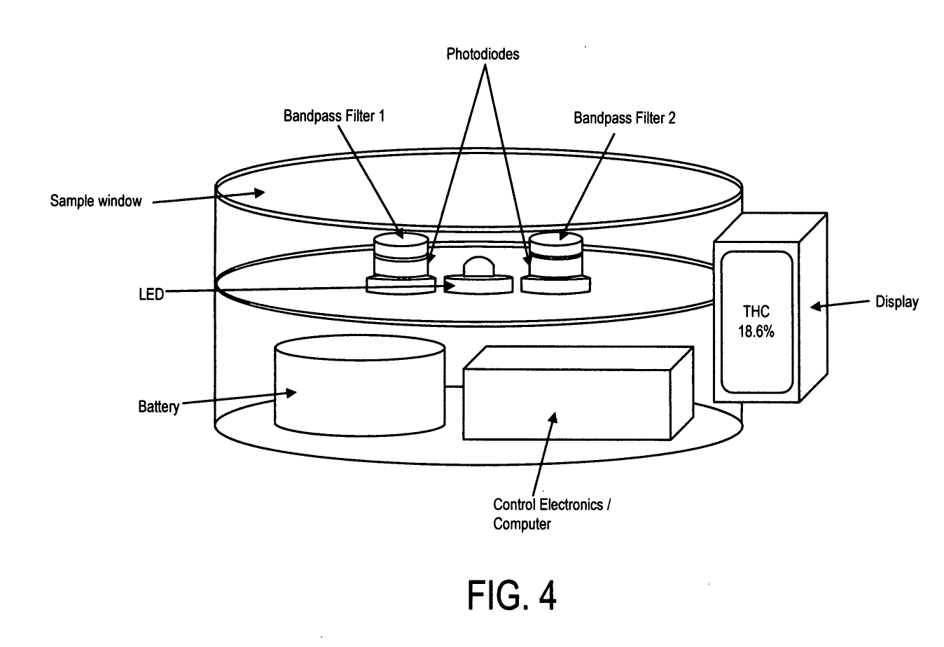

FIG. 3 illustrates a system that may be utilized for a characterized process, employing an embodiment of the monitoring apparatus of FIG. 1. The characterized process may have been characterized with the system described above with regard to FIGS. 1 and 2 in some embodiments. In the embodiment shown in FIG. 3, the monitoring apparatus 302 includes an optical measuring device 308 and a probe 304. Measuring device 308 and probe 304 may be flexibly coupled using fiber optics, for example. A light source 310 provides light to the probe 304, which passes the light to the sample 106. Light from the sample 106, which could be reflected light or induced fluorescence, or transmitted light in some configurations, passes from the sample 106 to the probe 304 and then to an optical module 306 in an optical measuring device 308. It should be appreciated that optical measuring device 308 may be referred to as the spectral engine discussed above with reference to FIG. 1A.

In a variation, the light source 310 could be inside the probe 304. In a further variation, the optical module 306 could be inside the probe 304. In some embodiments, the light source 310 is modular and can be readily swapped with other light sources as modules. In some embodiments, the optical module 306 is modular and can be readily swapped with other optical modules 306. In other embodiments, monitoring apparatus 302 is a modular apparatus that can be swapped based on the application. For example, a light source module could include one specific light source 310, and a matching optical module 306 could include one specific optical filter, matched to the light source 310, or could include several optical filters, matched to the light source 310 or could include a spectral engine as described in FIG. 1A. The light source 310 may be the optimum light source wavelength as identified through a survey of different light source wavelengths with the system of FIGS. 1 and 2 in some embodiments. In some embodiments, the monitoring apparatus 302 can be changed or swapped when different products are being manufactured. Each monitoring apparatus 302 can be configured for a specific process in some embodiments. The emission light is received by the detection unit 302 and may be communicated externally via the communication module 314, for example as a univariate (single variable) output to a storage device 316 or other suitable device or to display devices. The communication module 314 in various embodiments could communicate via a network, or wirelessly, and could communicate to various devices such as telephonic devices, computing devices, display devices and so on. In some embodiments, the communication module 314 is an embedded processor. In some embodiments, the storage device 316 is internal to the detection unit 302, or may be part of the embedded processor. The embodiment of FIG. 3 is suited for hazardous or not easily accessible environments, particularly since communication to remote devices is provided.

The storage device 316 can also be used to store characterization or calibration data of samples. For example, an operator of the detection unit 302 and probe 304, or other embodiment of the monitoring apparatus 102, could characterize samples or control portions having known amounts of an ingredient, or moisture level or other process parameter, and produce data showing a fluorescence peak, an absorption trough, or other spectral behavior that can be correlated with the process parameter. The characterization or calibration data could be stored in the storage device 316 or other memory, e.g., in tables correlating with the process parameter. Then, during manufacturing, the monitoring apparatus 102 can produce data relating to the spectrum of light from the sample, which data can be compared to the stored data. The operator could set ranges, trigger points, or other process control parameters based on the stored data and the comparison, and this could be used during process control and manufacturing. For example, a mixing process or a drying process could be stopped when a process parameter reaches a set point as determined by the analysis of data from the monitoring apparatus 102. It should be appreciated that the embodiments enable the in-line capture of the data while the manufacturing is progressing and a decision can be made in real time as opposed to stopping the process to pull one or more samples of the product to be tested off line to determine if the processing or particular step of the processing is complete, i.e., has reached an endpoint.

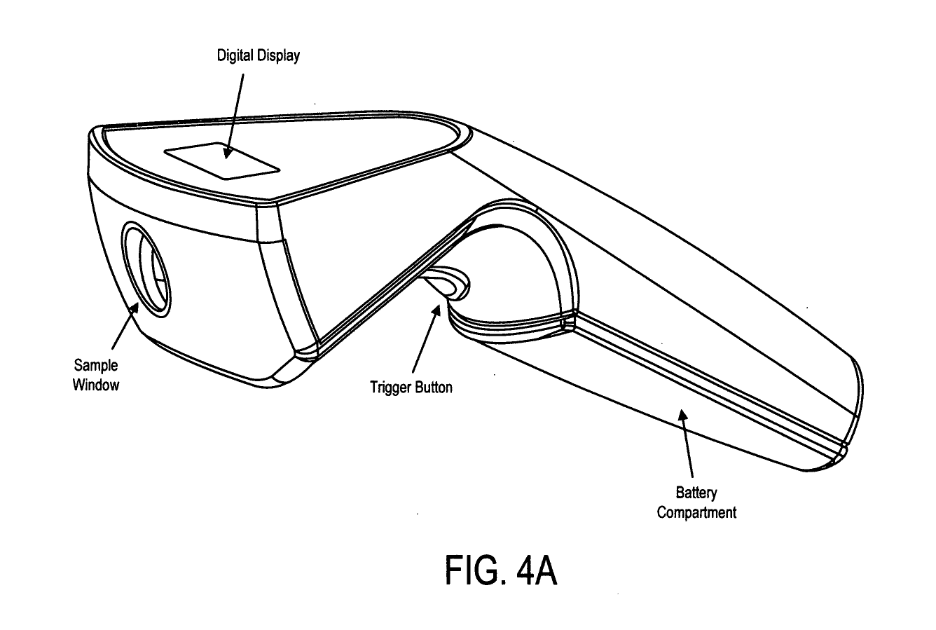

The system for on-site and portable measurement of cannabis of the present invention includes a photometer device that utilizes fluorescence of the cannabinoids for their quantification. The device is depicted in FIG. 4. As an illumination source, this device contains one or more LEDs with a peak emission wavelength between 100 and 2500 nm, more generally at 200 to 1000 nm, and in a preferred embodiment between 385 to 400 nm using certain standard operating equipment. The emission wavelength bandwidth is between 0.001 and 100 nm, but typically between 10 and 40 nm. The illumination source may contain an added wavelength band-pass filter to limit its emission wavelengths. There may be one or more illumination sources present, to allow illumination at the wavelengths and power levels necessary to generate a sufficient fluorescent signal. Multiple illumination sources may emit at the same wavelength to provide additive optical power or to provide more even illumination of the sample or to illuminate a larger area of a sample. Multiple illumination sources may be at different wavelengths to quantify different constituent compounds in the sample. The illumination geometry may vary, from a tightly focused spot of light as small as 0.01 mm to as large as 1 m, via a single emitter or a plural number of emitters. This geometry allows for a change in the interrogated sample area so the structures of interest can either be isolated or homogenized, as desired. For example, for microscopic study of individual cannabis trichomes it would be necessary to focus the illumination light down to a spot nearly the same size as the trichome. Alternately, to obtain an average measurement of the cannabinoid content across an entire plant, the illumination spot could be broadened to approximately the size of the plant. Another embodiment would allow for a small spot to be scanned over a larger area to allow measurement over the larger area but with higher irradiance than by defocusing one source to a larger area. This scanning could be accomplished using moveable mirrors, prisms, or similar beam-deflecting optics, including micro-optoelectronic machines (MOEMS).

In some embodiments, the device may contain optical elements such as lenses or optical fibers to direct the illumination and fluorescent light between the device and the sample. In other embodiments, the device may use a transparent interface or window to separate the sample from the emitters and detectors. The sample is touched to the window during measurement. In other cases, the sample is placed in a sample cup for presentation to the device. In other cases, a clip attached to the device is used to press the sample to the window. In yet other embodiments, the sample does not touch the device, as the optical elements are focused at some distance from the window. The sample may be any cannabis product or derivative containing cannabinoids, such as flowering buds, leaves, hash, oils, and extracts, among others.

As a detector, this device contains two silicon photodiodes. In other embodiments, this device utilizes other optical detectors such as photo-multiplier tubes, CCDs, CMOS cameras, thermal detectors, gallium detectors, or indium gallium arsenide detectors. The device may contain one or more detectors. In this embodiment of the device, the detectors each contain a wavelength band-pass filter to limit their respective collected wavelengths. In other embodiments, the detectors may contain other wavelength-selective elements such as gratings, prisms, or Fabry-Perot etalons. In some embodiments, the wavelength selective filters may be accomplished by depositing optical coatings directly on other optical elements in the device. As with the emitters, the collection area of the detector(s) could be directed to a larger area (1 m diameter) or a smaller area (0.01 mm diameter), depending on the intended interrogation. Also, like the emitters, this area can be accomplished by optical design to capture light from one large area or by scanning a smaller collection area across a larger region. Both the emitters and detectors are powered electronically by either line voltage or via batteries. Both the emitters and detectors are controlled and electrically filtered by external circuitry, to maximize their wavelength and intensity stability. In other cases, these devices do not contain external circuitry. The device contains a controlling microcomputer to sequence the emitters and the detectors, and to synchronize the collection of light from the sample. This sequencing can also be used to acquire an ambient reading before or after the sample reading to minimize optical noise from the sample environment. The illumination LED and the photodetectors may also be operated at specific duty cycles or flash rates to achieve modulation of power as may be necessary. Additionally, the emitters and detectors may be synchronized in such a way as to minimize interference from other light sources which may be present in the area where the instrument is used. In other embodiments, this controlling microcomputer may reside in an external computer or tablet, which communicates to the device via WiFi, Bluetooth, Ethernet, or a dedicated electronic connection or cord. The device contains a display to communicate with the user. In other embodiments, this display is an external computer or tablet. The displayed information provides the user with knowledge of the system status and operating procedures. The displayed information provides the user with the computed constituent values for the cannabinoids. In other embodiments, this information is not displayed to the user, but is printed on a label.

Such devices can be readily produced using well established circuit assembly techniques at low cost. The simplicity of LEDs as light sources is cost effective while providing excellent spectroscopic features. The small size and lower power requirements of the invention as described allow portable and low cost options to be realized. This is in direct contrast to most analytical systems currently available for cannabinoid testing. The simplicity and robustness of the invention enable ‘point and shoot’ measurements of cannabinoid concentrations by unskilled device operators, features that have been unavailable in the prior art.

In another embodiment, the device could be a part of, or an accessory for an electronic portable device such as a handheld mobile telephone, tablet device, smartphone, wrist or other device (collectively referred to as EP Device). The emitter for the measurement device could be the same emitter used on the EP Device as a flash for the camera or a range-finder for the autofocus mechanism. Or the illumination could come from the EP Device screen, using its inherent wavelength tunability and color palette. For example, if the EP Device screen were used as the illumination source, a particular color range could be emitted in the desired range to illuminate the sample, then the screen could be changed to emit a different color range to illuminate the sample. In another form, the illumination emitter could comprise a hardware device that could attach to the EP Device via WiFi, Bluetooth, Ethernet, or a dedicated electronic connection or cord. The detector for the measurement device could be the same camera incorporated into the EP Device, either in front-facing or rear-facing orientation. In another form, the measurement device detector could comprise a hardware device that could be attached to the EP Device via WiFi, Bluetooth, Ethernet, or a dedicated electronic connection or cord. Since these camera detectors are multi-elemented devices with numerous pixels, the same detector could be segmented electronically to allow detection of different wavelengths from different areas on the detector. A software application for the measurement device would allow implementation of the hardware for the intended method of cannabinoid quantification. This software application would coordinate the emitters and the detectors to allow synchronized operation, thereby minimizing extraneous light from ambient conditions and improving signal to noise ratio. The software application could also contain the necessary processing algorithm to translate the optical measurement into a cannabinoid quantity for ready presentation to the user. An example of a hand held portable device that incorporates embodiments of the present invention is shown in FIG. 4A. The hand held photometer device for cannabis measurement as described herein includes a window where light emanates to shine upon a sample, a trigger button to initiate the measurement sequence, and a digital display where a measurement reading is displayed. It is understood that a battery compartment is shown in the representative illustration, however such a device may be electrically powered through a cord and power outlet. It is also understood that wireless of cable connection of such a device to a computer allows a user to read and store measurements on a computer, or other electronic and storage device.

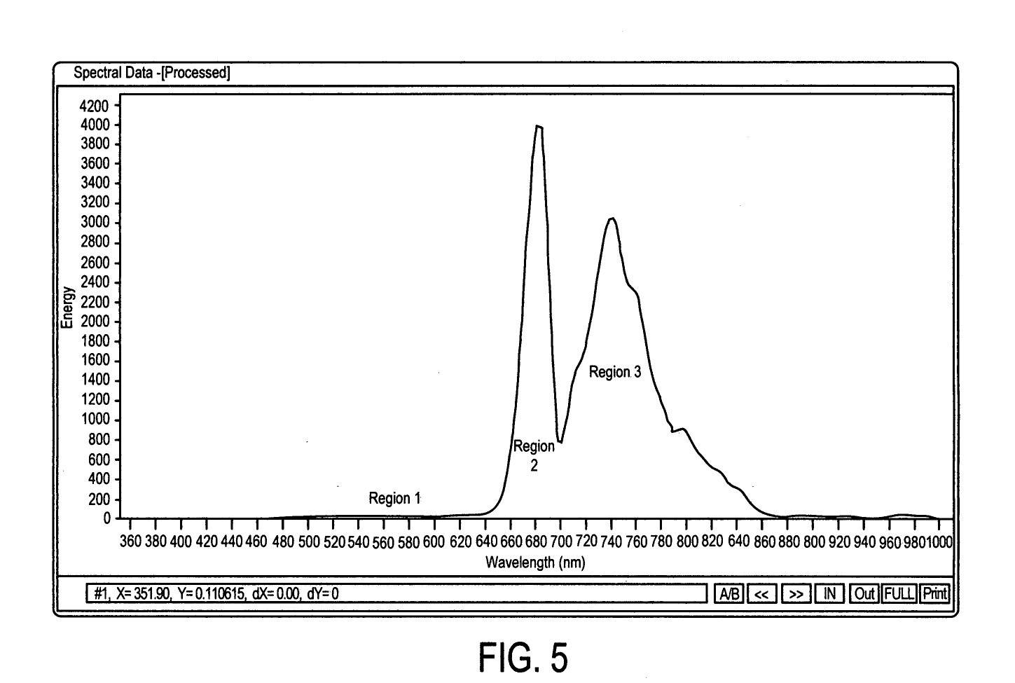

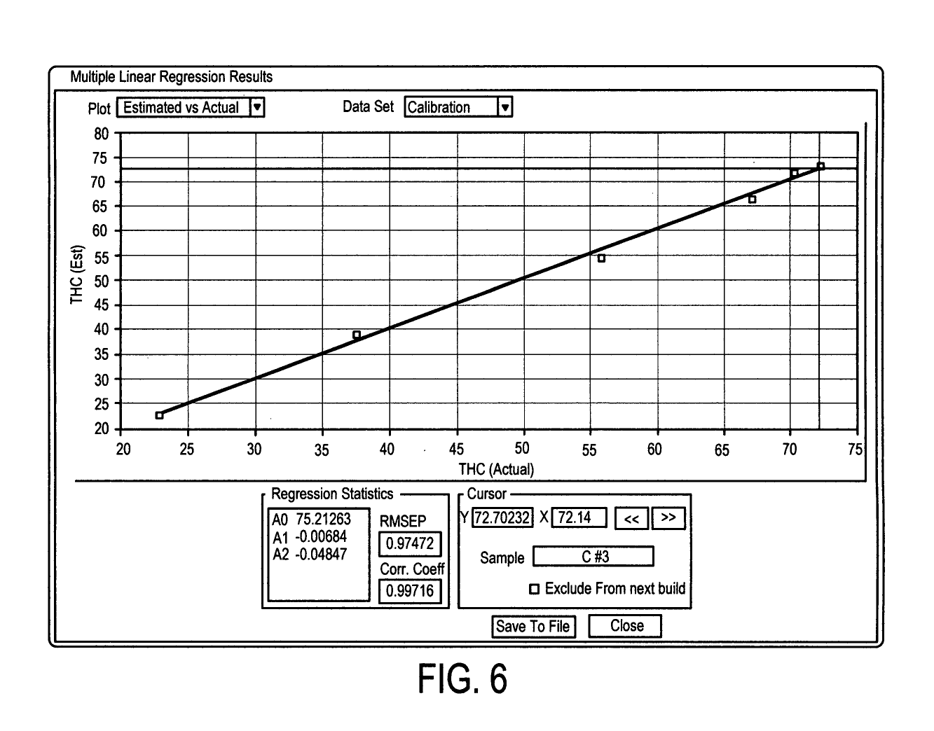

The method of the present invention entails the usage of the intrinsic fluorescence of the cannabinoids to permit their detection and quantification. Before measuring, the measurement device could be referenced to a stable standard with a generally uniform spectral response across the measurement range, such as PTFE, Spectralon, or lactose, to minimize artifacts from non-sample optical features. A representative fluorescence spectrum of a hash oil sample with high cannabinoid THC content is shown in FIG. 5. This spectrum was generated using a 385 nm excitation LED emitter for illumination. As seen in this spectrum, there are three primary spectral emission features that can be used for model development and cannabinoid quantification. The first region is the wavelength space between 400 and 600 nm, which is a low, broad feature. The second region is the distinct intensity peak between 600 and 700 nm. The third region is the large intensity peak and shoulders between 690 and 900 nm. The predictive model to correlate the fluorescence features with cannabinoid content may utilize either the intensity maxima at each of the three regions, or it may use the average value for each region, or it may use the area under each peak. In the case of THC quantification, only the first two peaks are utilized. FIG. 6 shows the regression model for THC in hash oil across a range of 23 to 72% that utilizes the peak intensity of the second region and the ratio of this intensity with the peak intensity of the first region in a multiple linear regression to predict THC content with a root-mean-square error less than 1%. Other cannabinoids such as CBD, CBN, and CBG could be classified in similar fashion, using a different regression model to correlate the fluorescence features with each cannabinoid quantity. Thus it is understood that measurements of moisture and other sample attributes are possible through the apparatus and method of the present invention.

As is well known in fluorescence spectroscopy an excitation-emission ‘map’ may be constructed to determine the optimum excitation wavelength producing the maximum emission for a given sample material. In the example shown in FIG. 5, the excitation wavelength region was measured from approximately 280 nm to 385 nm. At each excitation wavelength, the complete fluorescence spectrum was measured. FIG. 5 illustrates one such emission spectrum, where the fluorescence spectrum was optimally produced with 385 nm excitation. This procedure can be repeated for other cannabinoids with a range of excitation and emission wavelengths. This scanning procedure can be used to optimize the detection of the species of interest while minimizing signal from chemistries that are not of interest.

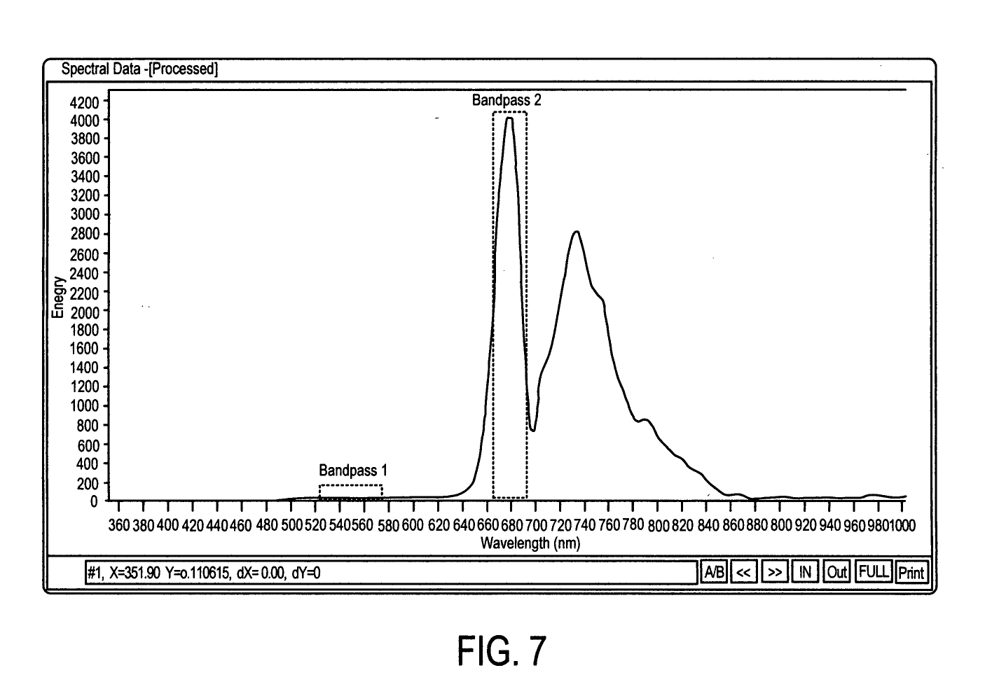

The method in the described invention would measure these intensity values using discrete detectors, such as described in the device details. As shown in FIG. 7, wavelength band-pass filters could be used on each of the two preferred detectors with pass bands as indicated. Thus, the optical intensities measured by each detector would equal the summed intensity under each of the respective curves, to the extent bounded by the filter pass bands. Thus the method employed by the preferred embodiment of the device would utilize a 385 nm LED emitter to excite the sample, one detector would measure the summed intensity between 525 and 575 nm, and the other detector would measure the summed intensity between 665 and 685 nm. These two integer values would be used in a similar regression model to output the THC percentage in the sample.

In the described method, a portion of the sample is placed in the measurement area of the instrument. No preparation of the sample is required. This feature is in contrast to other measurement systems, where the sample may need to be ground or powdered for measurement potentially precluding benefit of the sale and/or use of the sample after testing. Other measurement technologies may be destructive to the sample, destroying some or all quantities in the course of the measurement process. When the sample is in the measurement area, such as a cup or clip described earlier, or placed on the measurement window, the operator then presses a measurement button on the device or some similar means to indicate the sample is ready to be measured. Within a range of approximately 1 second to several seconds, a processor inputs the measured fluorescence amplitude values in each respective wavelength range into the predefined mathematical regression model to compute the cannabinoid concentration for the sample so it can be displayed to the user; as in FIG. 6. The sample is not damaged in any way during this measurement process. The display reading may show digitally on the device, or alternatively it may display remotely on a supplemental device, and/or may be physically printed for association with the product from which the sample belongs.

In contrast to other analytical methods currently employed, the device, system, apparatus and method described herein is easily executed by relatively unskilled operators. In particular, little to no knowledge of the spectroscopy used in the measurement is required. Little to no careful sample preparation is required for an accurate measurement, thereby enabling non-technical users to generate accurate measurements of cannabinoid species content therein, such species including but not limited to THC, CBD, CBN, CBG. Other features or properties of the cannabis plant as are currently known or may become known as advantageous to obtain accurate measurement such as moisture content may be measured by the system, device and methods described herein.

As used herein, the singular forms “a”, “an” and “the” are intended to include the plural forms as well, unless the context clearly indicates otherwise. It will be further understood that the terms “comprises”, “comprising”, “includes”, and/or “including”, when used herein, specify the presence of stated features, integers, steps, operations, elements, and/or components, but do not preclude the presence or addition of one or more other features, integers, steps, operations, elements, components, and/or groups thereof. Therefore, the terminology used herein is for the purpose of describing particular embodiments only and is not intended to be limiting.

Although the method operations were described in a specific order, it should be understood that other operations may be performed in between described operations, described operations may be adjusted so that they occur at slightly different times or the described operations may be distributed in a system which allows the occurrence of the processing operations at various intervals associated with the processing.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the embodiments and its practical applications, to thereby enable others skilled in the art to best utilize the embodiments and various modifications as may be suited to the particular use contemplated.

1. A method of nondestructively quantifying cannabinoids, comprising the steps of:
selecting a light source;
selecting an optical filter;
applying the light source to illuminate a sample of cannabinoid,
wherein upon illumination, light is produced by fluorescence of the sample and directed from the sample to the optical filter; and
quantifying the cannabinoid sample from the light directed to the optical filter.

2. The method of claim 1, further comprising the steps of:
removing at least a portion of the light directed from the cannabinoid sample to the optical filter;
wherein the optical filter filters the portion of light removed; and
measuring the intensity of the filtered light to measure at least one wavelength of light.

3. The method of claim 2, further comprising the step of:
correlating the measured intensity of light for the at least one wavelength of light from the cannabinoid sample, with a process variable of the sample.

4. The method of claim 1, wherein the sample quantified is a flowering bud.

5. The method of claim 1, wherein the sample quantified is ground plant.

6. The method of claim 1, wherein the sample quantified is a plant liquid extract.

7. The method of claim 1, wherein the light source has a peak emission wavelength between 385 and 400 nanometers.

8. The method of claim 1, wherein the light source has an emission wavelength bandwidth between 10 and 40 nanometers.

9. An apparatus for nondestructive quantification of cannabinoids, comprising:
a light source, wherein the light source illuminates a cannabinoid sample with light;
an optical filter, wherein the light illuminated on the cannabinoid sample is filtered to decrease intensity in at least one wavelength; and
an optical measuring device, wherein the device measures the wavelength and amplitude of light filtered from the optical filter and as correlated with a process variable of the cannabinoid sample.

10. The apparatus of claim 9, wherein the optical measuring device is an optical detector, photodiode, or charge coupled device.

11. The apparatus of claim 9, wherein the light source has a peak emission wavelength between 385 and 400 nanometers.

12. The apparatus of claim 9, wherein the light source has an emission wavelength bandwidth between 10 and 40 nanometers.

13. The apparatus of claim 9, wherein the light source illuminates the cannabinoid sample such that light is produced by fluorescence of the cannabinoid sample.

14. The apparatus of claim 9, wherein the apparatus comprises a photometer device.

15. The apparatus of claim 9, wherein the apparatus comprises a hand-held portable photometer device including a window through which light from the light source emanates to illuminate the cannabinoid sample, a trigger button to initiate a measurement sequence, and a digital display for displaying a measurement reading.

16. The apparatus of claim 9, wherein the apparatus comprises a photometer device including one or more photodiodes having one or more wavelength suppression filters to enable optical detection at one or more discrete wavelength bands.

17. A system for on-site, portable, and nondestructive quantification of cannabis, the system comprising a photometer device configured to utilize fluorescence of a cannabinoid sample for quantification, the photometer device including:
one or more light sources for illuminating the cannabinoid sample such that light is produced by intrinsic fluorescence of the cannabinoid sample; and
one or more detectors for detecting the light produced by the intrinsic fluorescence of the cannabinoid sample;
whereby the system is operable for detecting and quantifying the cannabinoid sample from the light produced by the intrinsic fluorescence of the cannabinoid sample detected by the one or more detectors.

18. The system of claim 17, wherein the photometer devices comprises a portable hand-held photometer device including a window through which light from the one more light sources emanates to illuminate the cannabinoid sample, a trigger button to initiate a measurement sequence, and a digital display for displaying a measurement reading.

19. The system of claim 18, wherein:
the one or more light sources comprises one or more LEDs having a peak emission wavelength between 385 and 400 nanometers and an emission wavelength bandwidth between 10 and 40 nanometers; and/or
the one or more detectors comprise one or more photodiodes having one or more wavelength suppression filters to enable optical detection at one or more discrete wavelength bands.

20. The method of claim 1, wherein the steps of the method are performed on-site using a portable hand-held photometer device and without requiring destruction of the cannabinoid sample.
